(19) United States
(12) Patent Application Publication
(10) Pub. No.: US 2014/0180387 A1
(43) Pub. Date: Jun. 26, 2014

(54) STENT DELIVERY SYSTEM

(71) Applicants: STRYKER NV OPERATIONS LIMITED, Dublin (IE); STRYKER CORPORATION, Kalamazoo, MI (US)

(72) Inventors: Michael Khenansho, Modesto, CA (US); Huey Chan, San Jose, CA (US)

(73) Assignees: STRYKER NV OPERATIONS LIMITED, Dublin (IE); STRYKER CORPORATION, Kalamazoo, MI (US)

(21) Appl. No.: 14/104,906

(22) Filed: Dec. 12, 2013

Related U.S. Application Data
(60) Provisional application No. 61/745,196, filed on Dec. 21, 2012.

(51) Int. Cl. A61F 2/966

(52) U.S. Cl. CPC A61F 2/966
USPC 623/1.12

(57) ABSTRACT

A stent delivery system includes an elongate delivery member; a self-expanding proximal bumper disposed on a distal region of the delivery member in a radially contracted configuration, where the proximal bumper is biased to expand to a radially expanded configuration; a self-expanding stent disposed over the delivery member in a radially contracted configuration distal of the proximal bumper; and a sheath disposed over the stent, proximal bumper, and delivery member, and configured to constrain the proximal bumper and stent in their radially contracted configurations, where the proximal bumper, when in its radially expanded configuration, has a cross-sectional dimension greater than an inner diameter of the sheath, such that the proximal bumper contacts the sheath and prevents movement of the stent proximal of the proximal bumper, and such that, as the delivery member is moved distally relative to the sheath, the proximal bumper pushes the stent in a distal direction.

### RELATED APPLICATION DATA

The present application claims the benefit under 35 U.S.C. §119 to U.S. provisional patent application Ser. No. 61/745,196, filed Dec. 21, 2012. The foregoing application is hereby incorporated by reference into the present application in its entirety.

### FIELD

The present disclosure relates generally to medical devices and intravascular medical procedures and, more particularly, to devices and methods for delivering a stent to a target site in a blood or other body vessel.

### BACKGROUND

The use of intravascular medical devices has become an effective method for treating many types of vascular disease. In general, a suitable intravascular device is inserted into the vascular system of the patient and navigated through the vasculature to a desired target site. Using this method, virtually any target site in the patient's vascular system may be accessed, including the coronary, cerebral, and peripheral vasculature.

Medical devices such as stents, stent grafts, and vena cava filters are often utilized in combination with a delivery device for placement at a desired location within the body. A medical prosthesis, such as a stent for example, may be loaded onto a stent delivery device and then introduced into the lumen of a body vessel in a configuration having a reduced diameter. Once delivered to a target location within the body, the stent may then be expanded to an enlarged configuration within the vessel to support and reinforce the vessel wall while maintaining the vessel in an open, unobstructed condition. The stent may be configured to be self-expanding, expanded by an internal radial force such as a balloon, or a combination of self-expanding and balloon expandable.

Many delivery devices include sheaths or catheters, and delivery members having bumpers thereon to push and pull stents through the sheaths and catheters. A catheter may be bent while navigating through torturous vasculature. As a catheter is bent, the cross-section of the catheter at the point of the bend changes from circular to ovular, i.e., ovalizes. As a catheter ovalizes, bumpers on the delivery members may lose contact with stents and stents may disengage from bumpers.

A number of different stent delivery devices, assemblies, and methods are known, each having certain advantages and disadvantages. However, there is an ongoing need to provide alternative stent delivery devices, assemblies, and methods. In particular, as the material used to form stents becomes thinner, there is an ongoing need to provide alternative stent delivery devices that maintain the ability of bumpers on delivery members to engage the stents in a longitudinal direction. There is also an ongoing need for bumpers that maintain contact with stents even as a catheter containing the bumper and stent ovalizes.

### SUMMARY

In one embodiment of the disclosed inventions, a stent delivery system includes an elongate delivery member; a self-expanding proximal bumper disposed on a distal region of the delivery member in a radially contracted configuration, where the proximal bumper is biased to expand to a radially expanded configuration; a self-expanding stent disposed over the delivery member in a radially contracted configuration distal of the proximal bumper; and a sheath disposed over the respective stent, proximal bumper, and delivery member, and configured to radially constrain the proximal bumper and the stent in their radially contracted configurations, where the proximal bumper, when in its radially expanded configuration, has a cross-sectional dimension greater than an inner diameter of the sheath, such that the proximal bumper, when in its radially contracted configuration, contacts the sheath and prevents movement of the stent proximal of the proximal bumper, and such that, as the delivery member is moved distally relative to the sheath, the proximal bumper pushes the stent in a distal direction.

In some embodiments, the proximal bumper includes a radially-extending strut having a first end attached to the delivery member, and a stent-contacting member attached to a second end of the radially-extending strut, and where the radially-extending strut is biased to impose an outward radial force against the stent-contacting member. The stent-contacting member may have an arcuate shape and may form an arc of between 20 degrees to 180 degrees. In some other embodiments, the proximal bumper further includes a second radially-extending strut having a first end attached to the delivery member and a second end attached to the stent-contacting member, where the second radially-extending strut is biased to impose an outward radial force against the stent-contacting member. In such embodiments, the stent-contacting member may form an arc of more than 180 degrees.

The stent-contacting member may include a low-friction outer surface. Further, the radially extending strut may include a shape-memory material, such as nitinol. Moreover, the proximal bumper may be configured such that, if the sheath is subject to a bending force, a cross-sectional dimension defined by the proximal bumper expands, so that the proximal bumper remains in contact with the sheath. The proximal bumper may also include a perfusion opening that permits fluid to flow from a proximal side of the proximal bumper to a distal side of the proximal bumper. Alternatively or additionally, the proximal bumper may have a non-circular, ellipsoid cross-sectional shape.

In another embodiment of the disclosed inventions, the proximal bumper includes a braided tubular member having a distal portion configured to radially expand such that, when the proximal bumper is radially constrained by the sheath, the distal portion of the braided tubular member contacts the sheath.

In yet another embodiment of the disclosed inventions, the proximal bumper includes a tubular body portion coupled to the delivery member and a plurality of stent-contacting members extending radially outward from a distal end of the tubular body portion. In such embodiments, the tubular body and the plurality of stent-contacting members may be integrally formed.

In any of these embodiments, the stent delivery system may also include a self-expanding distal bumper disposed on the delivery member distal of the stent in a radially contracted configuration, where the distal bumper is biased to expand to a radially expanded configuration in which the distal bumper has a cross-sectional dimension greater than an inner diameter of the sheath, such that the distal bumper, when in its radially contracted configuration, contacts the sheath and prevents movement of the stent distal of the distal bumper. The stent delivery system, may also include a self-expanding middle bumper disposed on the delivery member within an interior of the stent in a radially contracted configuration, where the middle bumper is biased to expand to a radially expanded configuration, such that the middle bumper impart a radially outward force against an interior surface of the stent, thereby resisting movement of the stent relative to the delivery member. The middle bumper may include a high friction outer coating.

Other and further aspects and features of embodiments of the disclosed inventions will become apparent from the ensuing detailed description in view of the accompanying figures.

### BRIEF DESCRIPTION OF THE DRAWINGS

The drawings illustrate the design and utility of embodiments of the disclosed inventions, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments of the disclosed inventions and are not therefore to be considered limiting of its scope.

FIG. 1 is a side view of a stent delivery system constructed according to one embodiment of the disclosed inventions, with a distal region of the system shown in an inset.

FIGS. 2A-2C are respective schematic views of the distal region of a stent delivery system constructed according to one embodiment of the disclosed inventions, illustrating placement of a stent at a target site in a blood vessel.

FIG. 3 is a side schematic view of a delivery wire and a sheath constructed according to one embodiment of the disclosed inventions, with details of the bumper omitted for clarity.

FIGS. 4A and 4B are detailed longitudinal cross-sectional views of the various bumpers in FIG. 3.

FIGS. 4C and 4D are detailed perspective views of the various bumpers in FIG. 3.

FIGS. 5 and 6 are top views of unrolled stents constructed according to embodiments of the disclosed inventions.

FIG. 7 is a detailed longitudinal cross-sectional view of the various bumpers in FIG. 3.

FIG. 8 is a sequence of top views of the distal end of a stent delivery system constructed according to one embodiment of the disclosed inventions, showing the delivery wire is being rotated 360 degrees about its longitudinal axis.

FIG. 9 is a sequence of top views of the distal end of a stent delivery system constructed according to one embodiment of the disclosed inventions, showing the stent being re-sheathed.

### DETAILED DESCRIPTION OF THE ILLUSTRATED EMBODIMENTS

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Various embodiments of the disclosed inventions are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. An aspect or an advantage described in conjunction with a particular embodiment of the disclosed inventions is not necessarily limited to that embodiment and can be practiced in any other embodiments even if not so illustrated.

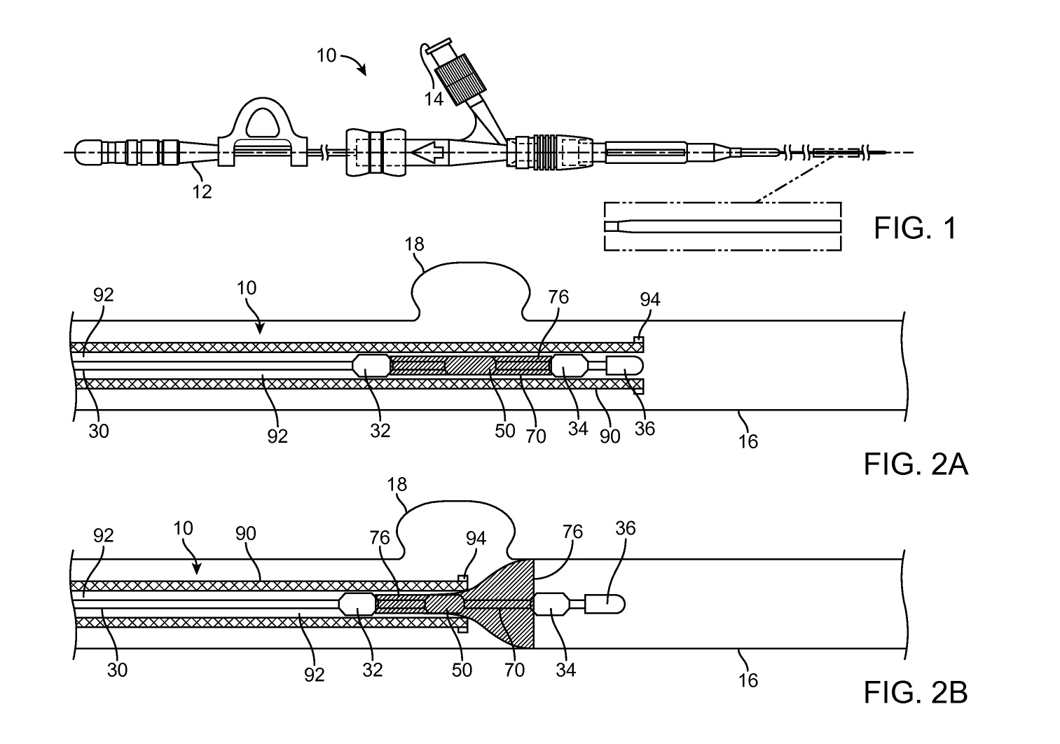

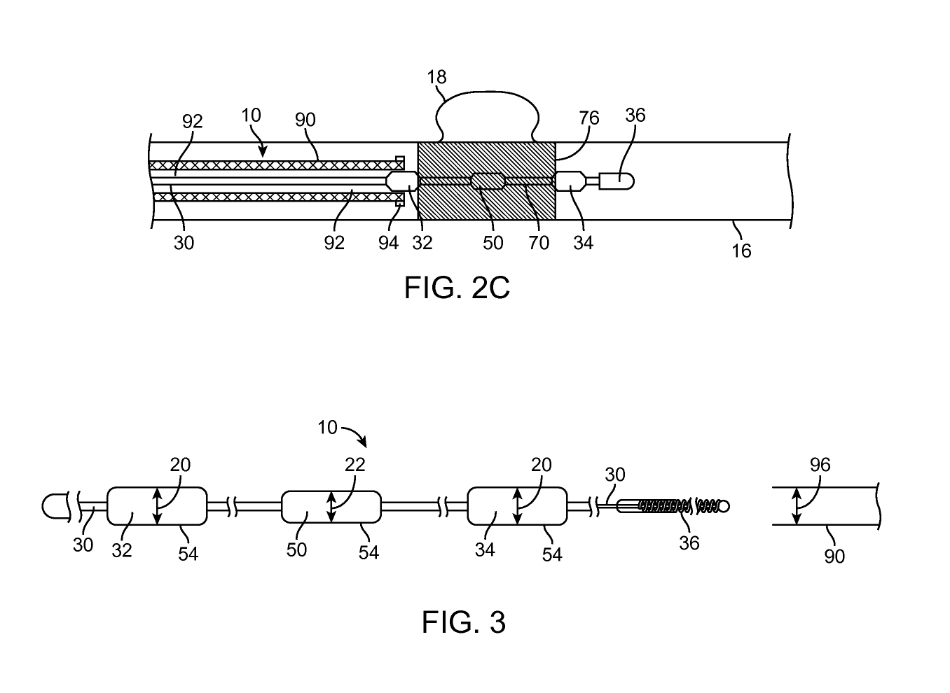

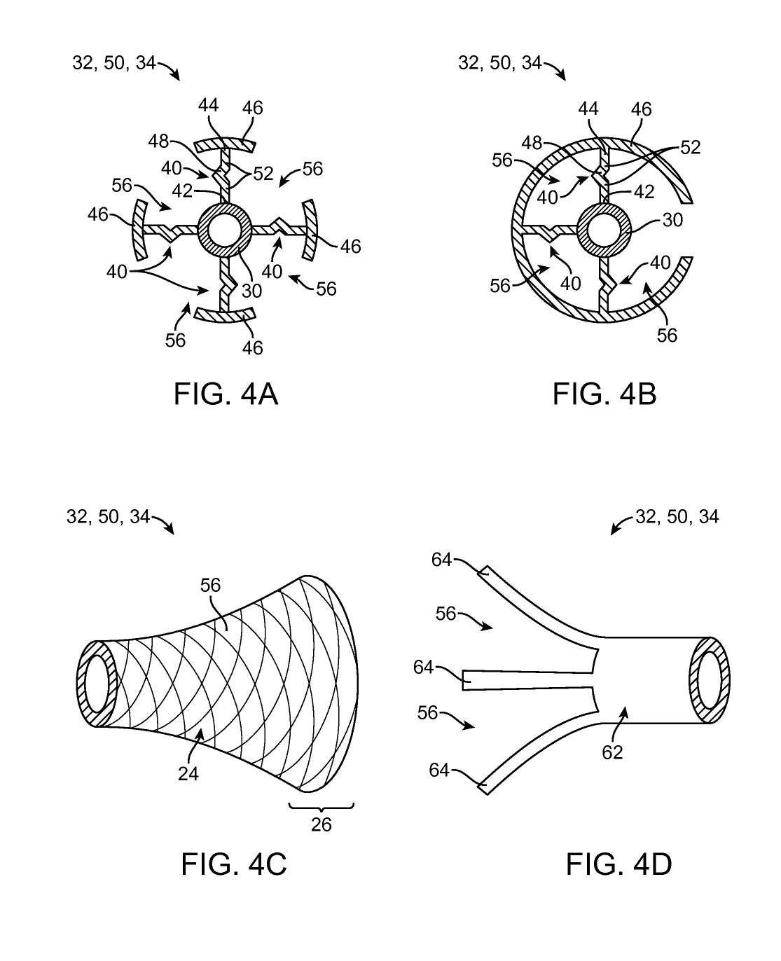

Referring to FIG. 1, the stent delivery system 10 has a handle 12 at its proximal end, which remains outside of the patients and accessible to the operator, when the system 10 is in use. The stent delivery system 10 also has a liquid port 14, which is used to introduce liquid into the system 10 to hydrate a stent 70 mounted therein. FIG. 2A is a schematic view of the stent delivery system 10, having a delivery member 30, a proximal bumper 32, a middle bumper 50, a distal bumper 34, a stent 70, and a sheath 90. Further details regarding the proximal, middle, and distal bumpers 32, 50, 34 are depicted in FIGS. 4A-D and 7. These parts of the system 10 are located in the distal end of the stent delivery system 10 shown schematically in the inset in FIG. 1.

Still referring to FIG. 2A, the delivery member 30 is a delivery wire 30, which has a proximal bumper 32, a distal bumper 34, and a distal tip 36. As described below, the proximal and distal bumpers 32, 34 are configured to self-expand from a radially contracted configuration to a radially expanded configuration, in which they each have a cross-sectional dimension greater than an inner diameter of the sheath 90. When constrained in their radially contracted configurations by the sheath 90, the proximal and distal bumpers 32, 34 each exert a radially outward force on an interior surface of the sheath 90. The proximal and distal bumpers 32, 34 are configured so that this radially outward force is sufficient to prevent a stent 70 disposed between the bumpers 32, 34 from moving proximal of the proximal bumper 32 or distal of the distal bumper 34. At the same time, the proximal and distal bumpers 32, 34 are configured so that the radially outward force, while sufficient to prevent dislocation of the stent 70, does not generate unnecessary friction between the bumpers 32, 34 and the sheath 90.

The middle bumper 50 is also configured to self-expand from a radially contracted configuration to a radially expanded configuration. However, as depicted in FIG. 2A, the middle bumper 50 is disposed under the stent 70. Accordingly, the middle bumper 50 is configured so that it exerts a radially outward force on the stent 70 sufficient to frictionally engage the stent 70, while minimizing the friction between the stent 70 and the sheath 90 that may damage the stent 70 during delivery. While the embodiment depicted in FIGS. 2A-C has self-expanding proximal, distal, and middle bumpers 32, 50, 34, other embodiments (not shown) may have only one or two self-expanding bumpers (described below). Still other embodiments (not shown) may combine self-expanding bumpers with non-self-expanding bumpers. Also, while the bumpers 32, 50, 34 depicted in FIGS. 2A-C and 3 have a capsule-like shape, other embodiments (not shown) include bumpers 32, 50, 34 that are more disc-shaped. Moreover, while the bumpers 32, 50, 34 depicted in FIGS. 4A-D appear to have a generally circular cross-section, other embodiments (not shown) include bumpers 32, 50, 34 that have any cross-section, including irregular shapes, as long as at least one cross-sectional dimension is greater than the diameter of the sheath 90.

Delivery wire 30 may be an elongate member having a proximal end and a distal end. Delivery wire 30 may be made of a conventional guidewire, torqueable cable tube, or a hypotube. In either case, there are numerous materials that can be used for the delivery wire 30 to achieve the desired properties that are commonly associated with medical devices. Some examples can include metals, metal alloys, polymers, metal-polymer composites, and the like, or any other suitable material. For example, delivery wire 30 may include nickel-titanium alloy, stainless steel, a composite of nickel-titanium alloy and stainless steel. In some cases, delivery wire 30 can be made of the same material along its length, or in some embodiments, can include portions or sections made of different materials. In some embodiments, the material used to construct delivery wire 30 is chosen to impart varying flexibility and stiffness characteristics to different portions of delivery wire 30. For example, the proximal region and the distal region of delivery wire 30 may be formed of different materials, for example materials having different moduli of elasticity, resulting in a difference in flexibility. For example, the proximal region can be formed of stainless steel, and the distal region can be formed of a nickel-titanium alloy. However, any suitable material or combination of material may be used for delivery wire 30, as desired.

Delivery wire 30 may further include a distal shapeable or pre-shaped tip 36, which may have an atraumatic distal end to aid in delivery wire 30 advancement. In some cases, distal tip 36 may include a coil placed over a portion of a distal end of the delivery wire 30 or, alternatively, may include a material melted down and placed over a portion of the distal end of delivery wire 30. In some cases, the distal tip 36 may include a radiopaque material to aid in visualization. Although not shown in the Figures, it is contemplated that a distal end of delivery wire 30 may include one or more tapered sections, as desired.

Delivery wire 30 may optionally include one or more bands (not shown) in a distal region of delivery wire 30. Bands may be formed integrally into the delivery wire 30, or they may be separately formed from delivery wire 30 and attached thereto. In some cases, the bands may be disposed on delivery wire 30. The bands may have a diameter greater than the diameter of the surrounding delivery wire 30. Bands may be formed of any suitable material, such as metals, metal alloys, polymers, metal-polymer composites, and the like, or any other suitable material, as well as any radiopaque material, as desired. Alternatively, it is contemplated that the delivery wire 30 may include one or more recesses instead of providing bands, if desired.

As schematically depicted in their expanded configuration in FIG. 3, the proximal and distal bumpers 32, 34 have a cross-sectional dimension 20 greater than an inner diameter 96 of the sheath 90. The middle bumper 50 also has a cross-sectional dimension 22 in its expanded configuration, which is less than the cross-sectional dimension 20 of the proximal and distal bumpers 32, 34 to facilitate mounting of a stent 70 on the middle bumper 50. While the proximal and distal bumpers 32, 34 are depicted as having the same cross-sectional dimension 20, they can have different cross-sectional dimensions.

The bumpers 32, 50, 34 may be radiopaque, in which case they function as markers to facilitate determination of delivery wire position. The distal tip 36 is floppy and steerable using pull wires (not shown) to facilitate tracking of the stent delivery system 10 through a vessel 16 to reach a target site 18, such as an aneurysm 18.

Various embodiments of self-expanding bumpers, such as proximal, middle, and distal bumpers 32, 50, 34, are depicted in FIGS. 4A-D. In the embodiments depicted in FIGS. 4A and 4B, the self-expanding bumper 32, 50, 34 includes a radially-extending strut 40 having a first end 42 attached to the delivery member 30 and a second end 44 attached to a stent-contacting member 46. The radially-extending strut 40 has a bent section 48 surrounded by two straight sections 52. The bent section 48 is configured to be elastically compressible, for instance, by heat-setting a stainless steel or shape memory alloy (e.g., nitinol) radially-extending strut 30. The stent-contacting member 46 is arcuate to conform to the shape of an approximately tubular stent 70. Consequently, when the bumpers 32, 50, 34 depicted in FIGS. 4A and 4B are radially constrained by a sheath 90, the radially-extending struts 40 are compressed and impose an outward radial force against the stent-contacting member 46 and the sheath 90. The outward radial force pushes the stent-contacting member 46 into contact with the sheath 90, and allows an axial surface of the stent-contacting member 46 to contact a stent 70.

In the embodiment depicted in FIG. 4A, the self-expanding bumper 32, 50, 34 includes four radially-extending struts 40 respectively attached to four stent-contacting members 46. Each of the stent-contacting members 46 forms an arc of about 20 degrees. Other embodiments (not shown) have different numbers of radially-extending struts 40 and stent-contacting members 46. Alternatively or additionally, other stent-contacting members 46 (not shown) form arcs of between 20 degrees and 180 degrees or greater than 180 degrees. For instance, FIG. 4B depicts a self-expanding bumper 32, 50, 34 including a stent-contacting member 46 forming an arc of about 340 degrees. Three radially-extending struts 40 are attached to different locations on the stent-contacting member 46. The self-expanding bumpers 32, 50, 34 depicted in FIGS. 4A and 4B also include perfusion openings 56 that permit fluid to flow from a proximal side to a distal side of the bumpers 32, 50, 34.

In the embodiment depicted in FIG. 4C, the self-expanding bumper 32, 50, 34 is formed of a braided tubular member 24. In the expanded configuration, as depicted in FIG. 4C, a distal portion 26 of the braided tubular member 24 is radially expanded relative to the rest of the braided tubular member 24. Accordingly, when compressed by a sheath 90, the distal portion 26 is configured to contact the sheath 90, thereby allowing an axial surface of the distal portion 26 of the tubular member 24 to contact a stent 70.

The braided tubular member 24 may be formed by braiding together nitinol filaments or ribbons, and the shape in the expanded configuration may be heat-set. The braided structure provides perfusion openings 56 in the tubular member 24.

In the embodiment depicted in FIG. 4D, the self-expanding bumper 32, 50, 34 is formed of a tubular body 62 having a plurality of stent-contacting members 64 extending from the distal end of the tubular body 62. For example, the embodiment depicted in FIG. 4D has three stent-contacting members 64. In the expanded configuration, as depicted in FIG. 4D, the stent-contacting members 64 extend distally and radially outward from the distal end of the tubular body 62. Accordingly, when compressed by a sheath 90, the stent-contacting members 64 are configured to contact the sheath 90, thereby allowing an axial surface of each stent-contacting member 64 to contact a stent 70.

In another embodiment the middle self-expanding bumper 50 is the bumper 50 depicted in FIG. 4D. The bumper 50 has been flipped horizontally so that the three stent-contacting members 64 extend from the proximal end of the tubular body 62. In the expanded configuration, the stent-contacting members 64 extend distally and radially outward from the distal end of the tubular body 62. Accordingly, when compressed by a sheath 90 and an overlying stent 70, the stent-contacting members 64 are configured to contact the stent 70, thereby imparting a radially outward force against an interior surface of the stent 70. In this manner, the stent-contacting members 64 of the middle bumper 50 resist movement of the stent 70 in a distal direction relative to the delivery member 30. This design facilitates re-sheathing of a partially deployed stent 70, where the sheath 90 continues to compress the stent 70 and the stent-contacting members 64 in respective radially contracted configurations. In that configuration, the middle bumper 50 can be used to pull the stent 70 proximally relative to the sheath 90 to re-sheath the stent 70.

In some embodiments, the stent-contacting members 64 are sized to partially enter openings in the stent 70, thereby allowing a proximally facing axial surface of each stent-contacting member 64 to contact the stent 70. This design further resists movement of the stent 70 in a distal direction relative to the delivery member 30, further facilitating re-sheathing of a partially deployed stent 70.

The bumper 32, 50, 34 may be formed from a stainless steel or nitinol hypotube. The stent-contacting members 64 can be formed by removing sections of the hypotube between the stent-contacting members 64, which also forms perfusion openings 56 in the bumper 32, 50, 34. Then the stent-contacting members can be bent radially outward and heat-set.

In order to increase fluid flow from a proximal side to the distal side of the bumper 32, 50, 34, the portions of the bumper 32, 50, 34 configured to contact the sheath 90 may have a non-circular, ellipsoidal, cross-section. Such a cross-sectional shape is shown schematically in FIG. 7. This general shape can be applied to any bumper 32, 50, 34, such as the one depicted in FIGS. 4A-D.

The outer surface 54 of the proximal and distal bumpers 32, 34 may impart low-friction due to the material from which the proximal and distal bumpers 32, 34 are formed, e.g., metals like nitinol. Alternatively or additionally, the outer surface 54 of the proximal and distal bumpers 32, 34 may be coated with a lubricious coating (not shown), e.g., polytetrafluoroethylene (PTFE). A low-friction outer surface 54 facilitates movement of the proximal and distal bumpers 32, 34 through the sheath 90, while in contact therewith.

The outer surface of the middle bumper 50 may impart high friction due to a tacky outer surface 54, which may be formed by coating or covering the middle bumper 50 with a high-friction polymer, e.g. PEBAX. A high-friction, tacky outer surface 54 aids the middle bumper 50 in resisting movement of the stent 70 relative to the delivery member 30.

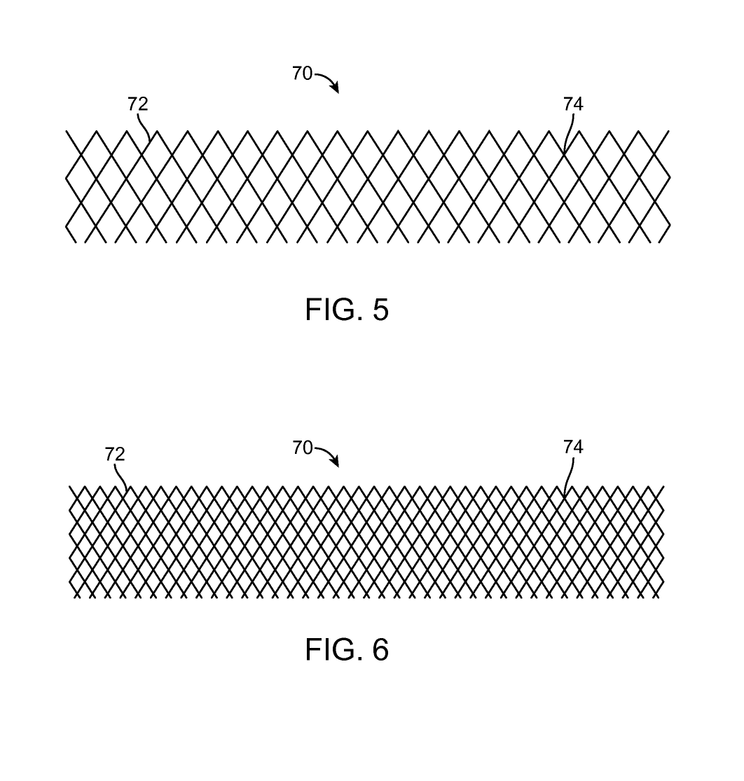

FIG. 5 illustrates a stent 70 for use with the stent delivery system 10. The stent 70 has a closed loop design in that adjacent ring segments 72 are connected at every possible junction 74. However, the stent delivery system 10 may be used with stents having other designs. The stent delivery system 10 may also be used with stents 70 having an overlapping or layered arrangement, as shown in FIG. 6. Overlapping stents 70 may increase the density of coverage or, in other words, decrease the porosity of the cellular configuration or pattern. The increase in the density of coverage may reduce the number of particles that may pass through the stent cells when in use. Such a feature may more effectively divert blood flow away from an aneurysm to help prevent the aneurysm from rupturing.

As illustrated in FIG. 6, the two layers of stent 70 may be longitudinally offset so that the cellular patterns do not completely overlap. For example, the layers may be longitudinally offset by about one-half cell length. However, the layers may be offset by about one-eighth cell length, one-quarter cell length, three-quarter cell length, or any other offset length, as desired. If, however, layers are not offset so that there is complete ring segment 72 overlap due to flow in the vessel or other factors, there may be no or relatively little increase in the density of coverage. Due to the varying degrees of coverage based on the offset or alignment of layers of stent 70, the stent 70 may have a relatively low density of coverage predictability. In some situations, stents having cellular configurations or patterns differing in at least one aspect may increase the predictability of the density of coverage of the assembly. For example, stents having different patterns, mirrored patterns (e.g., left-handedness, right-handedness), different periodicity of patterns, as well as stents of different constructions (e.g., tube, braid) or different materials may be used to help increase the predictability of the density of coverage or cellular porosity.

Further, it is contemplated that the stents 70 may be deployed in an overlapping or layered arrangement or, in other cases, may be interference fit, joined, or otherwise connected to form a multi-layer stent prior to deployment, as desired. In some cases, a single layer stent may be inverted prior to assembly, during deployment, or after deployment to form a multi-layer stent.

For merely illustrative purposes, the foregoing stents 70 have been shown in a flattened view or as a sheet. However, the stents 70 may be rolled into a generally tubular structure, similar to stent 70 shown in FIG. 2A, which may or may not have a generally varied cross-section.

The tubular stent 70 defines a lumen 76 representing the inner volumetric space bounded by the stent 70. The stent 70 is radially expandable from an unexpanded state (FIG. 2A) to an expanded state (FIG. 2C) to allow the stent 70 to expand radially and support the vessel 16. In the illustrative embodiments, the stent 70 is self-expanding. A sheath 90 or other device may be used to radially constrain the stent 70 while being delivered to a target site 18 within the body. When the sheath 90 or other device is retracted proximally from the stent 70, the stent radially expands to a second configuration having a larger diameter, as described in greater detail below.

Further, the foregoing stents 70 may be constructed of any number of various materials commonly associated with medical devices. Some examples can include metals, metal alloys, polymers, metal-polymer composites, as well as any other suitable material. Examples may include stainless steels, cobalt-based alloys, pure titanium and titanium alloys, such as nickel-titanium alloys, gold alloys, platinum, and other shape memory alloys. However, it is contemplated that the foregoing stents 70 may be constructed of any suitable material, as desired. In some cases, different layers of stents 70 may be constructed of different materials, if desired.

Additionally, the foregoing stents 70 may be delivered to a target site 18 by two separate delivery systems 10 to sequentially deliver the stents 70 or, in other cases, by a single multiple stent delivery system. In some cases, the multiple stent delivery system may have the stents 70 mounted thereon in an overlapping arrangement or in a tandem arrangement.

In the illustrative embodiments, the stent 70 may be disposed on a portion of the distal region of delivery wire 30 in a radially constrained first configuration. The stent 70 may be a self-expanding stent. In this example, the stent 70 may be radially constrained by sheath 90 while being delivered to a target site 18 within the body, but when sheath 90 is retracted proximally, the stent 70 may radially expand to a second configuration having a larger diameter.

The stent delivery system 10 includes a retractable sheath 90 disposed over the delivery wire 30 and stent 70. The sheath 90 may take the form of a catheter 90. The sheath 90 may be an elongate tubular member that may have a distal region or end that is disposed over delivery wire 30, having an annular space sufficient in size to receive the radially contracted stent 70 therein. The sheath defines a sheath lumen 92 extending between the proximal and distal ends. The lumen 92 of the catheter 90 is sized to accommodate longitudinal movement of the radially contracted stent 70, the middle bumper 50, and the delivery wire 30. In the illustrative embodiment, movement of sheath 90 in a proximal direction relative to delivery wire 30 may expose the stent 70, allowing expansion of the stent 70.

There are numerous materials that can be used for the sheath 90 to achieve the desired properties that are commonly associated with medical devices. Some examples can include metals, metal alloys, polymers, metal-polymer composites, and the like, or any other suitable material. Examples of suitable metals and metal alloys can include stainless steel, such as 304V, 304L, and 316L stainless steel; nickel-titanium alloy such as a superelastic (i.e., pseudoelastic) or linear elastic nitinol; nickel-chromium alloy; nickel-chromium-iron alloy; cobalt alloy; tungsten or tungsten alloys; tantalum or tantalum alloys, gold or gold alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); or the like; or other suitable metals, or combinations or alloys thereof. Examples of some suitable polymers can include, but are not limited to, polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), polyether-ester, polymer/metal composites, or mixtures, blends or combinations thereof. Sheath 90 can optionally be lined on an inner surface, an outer surface, or both with a lubricious material, if desired.

The catheter 90 may include a braided-shaft construction of stainless steel flat wire that is encapsulated or surrounded by a polymer coating. By way of non-limiting example, HYDROLENE® is a polymer coating that may be used to cover the exterior portion of the delivery catheter 90. Of course, the system 10 is not limited to a particular construction or type of catheter 90 and other constructions known to those skilled in the art may be used for the catheter 90.

The sheath lumen 92 may be advantageously coated with a lubricious coating such as PTFE to reduce frictional forces between the catheter 90 and the stent 70 being moved longitudinally within the lumen 92. The catheter 90 may include one or more optional marker bands 94 formed from a radiopaque material that can be used to identify the location of the distal end of the catheter 90 within the patient's vasculature system or relative to the proximal, middle, and proximal bumpers 32, 50, 34 using imaging technology (e.g., fluoroscope imaging).

As shown in FIG. 2A, the stent delivery system 10 may be positioned in the vessel 16 so that stent 70 is positioned adjacent to the target site 18, which in the illustrative example is a weakened region of the vessel 16 or an aneurysm 18. In some cases, the stent 70 may be configured to be deployed across the aneurysm 18 to help divert blood flow in the vessel 16 from entering the aneurysm 18. However, this treatment site is merely illustrative and is not meant to be limiting in any manner. It is contemplated that the delivery system 10 may be used to deliver stents to other target sites, such as stenoses, as desired.

In some cases, the sheath 90 and delivery wire 30 with radially contracted stent 70 may be advanced to the target site, or aneurysm 18, as an assembly. In these cases, the stent delivery system 10 may optionally be inserted into a proximal end of an introducer or other catheter and subsequently advanced to the aneurysm 18. In other cases, the sheath 90 may be advanced to the target site first and then the delivery wire 30 with radially contracted stent 70 may be inserted into a proximal end of sheath 90 and advanced through the sheath lumen 92 to the target site 18.

FIGS. 2A-2C are schematic views of an illustrative procedure for deploying a stent 70 in a vessel 16 using the stent delivery system 10 of FIG. 1. Preliminarily, the stent 70 is mounted between proximal and distal bumpers 32, 34, and around a middle bumper 50 attached to a delivery wire 30. Then the stent 70 and the delivery wire 30, with its proximal, middle, and distal bumpers 32, 50, 34 are threaded longitudinally into a sheath 90 using either an iris crimper (available from Machine Solutions, Inc.) or a funnel. Radial expansion of the contracted proximal and distal bumpers 32, 34 against the sheath 90 prevents movement of the stent 70 proximal of the proximal bumper 32 or distal of the distal bumper 34 as the stent 70 and delivery wire 30 are threaded through the sheath 90. Radial expansion of the contracted middle bumper 50 and its tacky outer surface 54 resists axial movement of the stent 70 relative to the middle bumper 50 and the delivery wire 30 during deployment and re-sheathing.

Next hydrating liquid, such as normal saline, is introduced into the liquid port 14 at the proximal end of the system 10. In some embodiments, such as the ones depicted in FIGS. 4A-D, the hydrating liquid travels through the proximal end of the sheath lumen 92 and the perfusion openings 56 in the proximal and middle bumpers 32, 50 to the distal end of the sheath lumen 92 to hydrate the stent 70. In the embodiment depicted in FIG. 7, the hydrating liquid travels around the proximal and middle bumpers 32, 50.

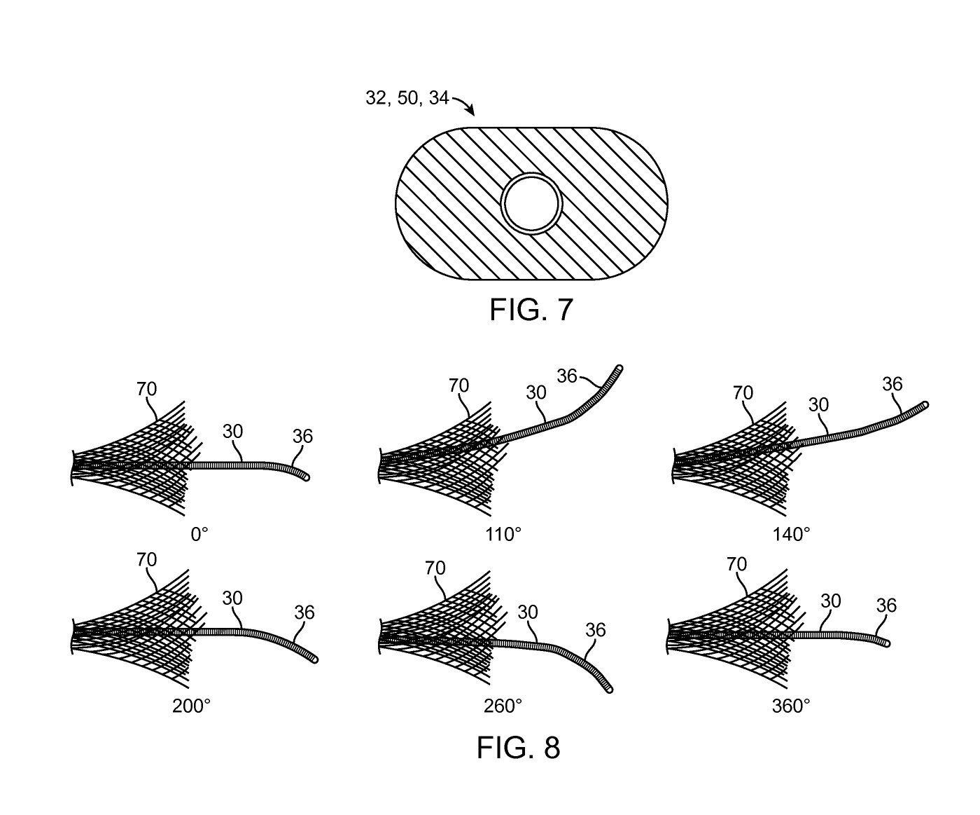

The distal end of the stent delivery system 10 is then introduced into a vessel 16 containing an aneurysm 18 and advanced to the aneurysm 18. The distal tip 36 of the delivery wire 30 may be steered to track the system 10 through the vessel 16. The stent 70 may also be moved relative to the sheath to position the stent 70 relative to the aneurysm 18. As the stent 70 is moved distally relative to the sheath 90, the proximal and middle bumpers 32, 50 cooperate to prevent the stent 70 from moving proximally. As the stent 70 is moved proximally relative to the sheath 90, the middle and distal bumpers 50, 34 cooperate to prevent the stent 70 from moving distally. The middle bumper 50 may be floating, i.e., disposed around, but not attached to the delivery wire 30. In embodiments with floating middle bumpers 50, the delivery wire 30 may torqued, or rotated about its longitudinal axis, to provide further tracking ability in addition to that provided by steering the distal tip 36. FIGS. 8A to 8F show delivery wire 30 torquing in such an embodiment.

When a sheath 90 is positioned in torturous vasculature, the sheath 90 may bend and ovalize, i.e., the cross-section of the catheter at the point of the bend changes from circular to ovular. Because the proximal and distal bumpers 32, 34 described above exert a radially outward force against the sheath 90, when the proximal and distal bumpers 32, 34 reach an ovalized portion of the sheath 90, they radially expand to maintain contact with the sheath 90. Maintaining contact with the sheath 90 reduces the incidence of stents disengaging from the proximal and distal bumpers 32, 34 when the sheath 90 ovalizes.

After the stent delivery system 10 has been positioned so that the stent 70 is aligned with aneurysm 18, as shown in FIG. 2A, sheath 90 is partially retracted from the delivery wire 30 to expose a distal portion of stent 70. As the sheath 90 is retracted, the proximal and middle bumpers 32, 50 cooperate to prevent the stent 70 from moving proximally. As illustrated in FIG. 2B, when self-expanding stent 70 is exposed, the stent 70 radially expands to engage a portion of the vessel 16 wall. Optionally, the relative positions of the distal end of the sheath 90 to the middle bumper 50 are monitored while retracting the sheath 90. Radiological visualization of the marker band 94 mounted at the distal end of the sheath 90 and the middle bumper 50 can be used to monitor their relative positions. Such positional monitoring avoids prematurely unsheathing the stent 70 over the middle bumper 50 and releasing the stent 70 from the middle bumper 50.

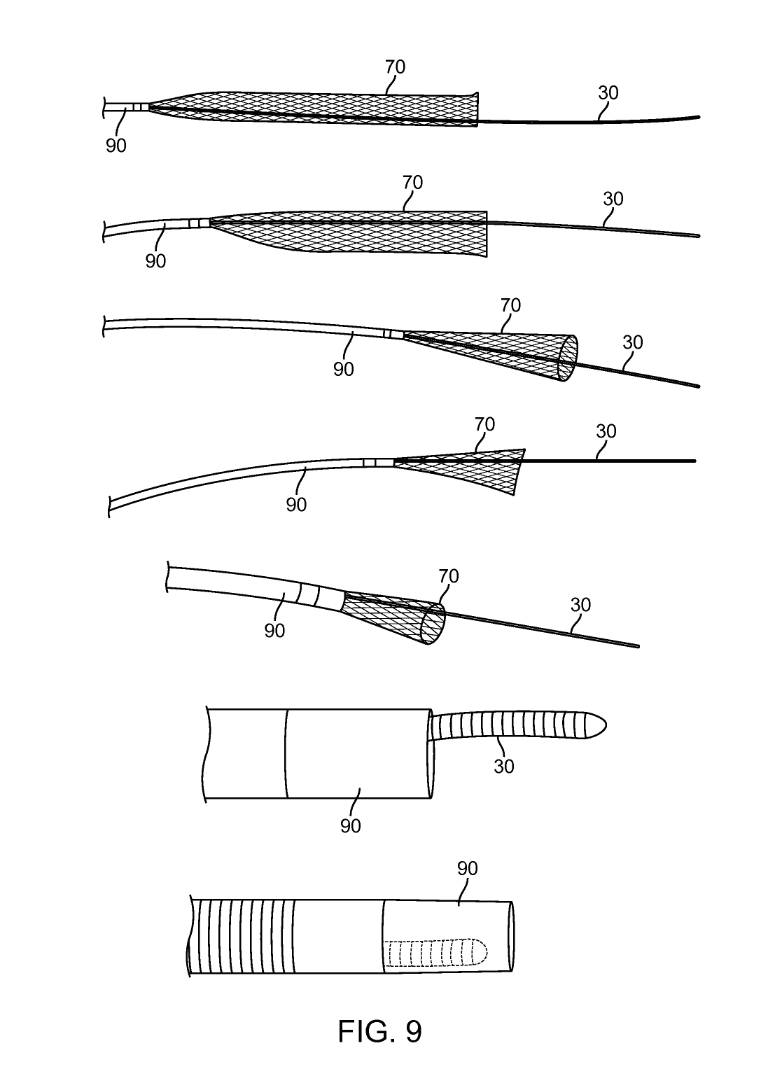

When the stent 70 is partially unsheathed, the position of the stent 70 relative to the aneurysm 18 is determined by radiological visualization. If the position of the partially unsheathed stent 70 is not correct, e.g., misaligned with the aneurysm, the stent 70 is re-sheathed by advancing the sheath 90 distally over the stent 70 or pulling the delivery wire 30 and the stent 70 by way of the middle bumper 50 proximally into the sheath 90. The re-sheathing process is shown in FIGS. 9A to 9G. Using the middle bumper 50 of the above embodiments of the disclosed inventions, an 80% unsheathed stent 70, such as the one shown in FIG. 8A, can be fully re-sheathed. After re-sheathing the stent 70, the sheath 90 and stent 70 contained therein are repositioned based on the previously determined position. The process of partially unsheathing, position determining, re-sheathing, and repositioning is repeated until the position of the partially unsheathed stent 70 relative to the aneurysm 18 is correct.

Next, as illustrated in FIG. 2C, continued retraction of sheath 90 relative to delivery wire 30 to a position proximal of stent 70 completely deploys stent 70. As stent 70 is deployed, the stent 70 fully expands and engages the vessel 16 wall on both sides of aneurysm 18. The stent 70 also expands away from the middle bumper 50.

With stent 70 deployed, delivery wire 30 and middle bumper 50 may be optionally retracted into sheath 90. Then, sheath 90, middle bumper 50, and delivery wire 30 may be withdrawn from the vessel 16 together.

In some embodiments, a degree of MRI compatibility is imparted into catheters. For example, to enhance compatibility with Magnetic Resonance Imaging (MRI) machines, it may be desirable to make the stent delivery system 10 or other portions of the stent delivery system 10 in a manner that would impart a degree of MRI compatibility. For example, delivery wire 30, middle bumper 50, stent 70, sheath 90, or other portions of the stent delivery system 10 may be made of a material that does not substantially distort the image and create substantial artifacts (artifacts are gaps in the image). Certain ferromagnetic materials, for example, may not be suitable because they may create artifacts in an MRI image. Stent delivery systems 10 or portions thereof may also be made from a material that the MRI machine can image. Some materials that exhibit these characteristics include, for example, tungsten, Elgiloy, MP35N, nitinol, and the like, and others. In some embodiments, a sheath and/or coating, for example a lubricious, a hydrophilic, a protective, or other type of material may be applied over portions or all of the stent delivery system 10 or other portions of the system 10.

Although particular embodiments of the disclosed inventions have been shown and described herein, it will be understood by those skilled in the art that they are not intended to limit the present inventions, and it will be obvious to those skilled in the art that various changes and modifications may be made (e.g., the dimensions of various parts) without departing from the scope of the disclosed inventions, which is to be defined only by the following claims and their equivalents. The specification and drawings are, accordingly, to be regarded in an illustrative rather than restrictive sense. The various embodiments of the disclosed inventions shown and described herein are intended to cover alternatives, modifications, and equivalents of the disclosed inventions, which may be included within the scope of the appended claims.

What is claimed is:

1. A stent delivery system, comprising:
an elongate delivery member;
a self-expanding proximal bumper disposed on a distal region of the delivery member in a radially contracted configuration, wherein the proximal bumper is biased to expand to a radially expanded configuration;
a self-expanding stent disposed over the delivery member in a radially contracted configuration distal of the proximal bumper; and
a sheath disposed over the respective stent, proximal bumper, and delivery member, and configured to radially constrain the proximal bumper and the stent in their radially contracted configurations,
wherein the proximal bumper, when in its radially expanded configuration, has a cross-sectional dimension greater than an inner diameter of the sheath, such that the proximal bumper, when in its radially contracted configuration, contacts the sheath and prevents movement of the stent proximal of the proximal bumper, and such that, as the delivery member is moved distally relative to the sheath, the proximal bumper pushes the stent in a distal direction.

2. The stent delivery system of claim 1, wherein the proximal bumper comprises a radially-extending strut having a first end attached to the delivery member, and a stent-contacting member attached to a second end of the radially-extending strut, and
wherein the radially-extending strut is biased to impose an outward radial force against the stent-contacting member.

3. The stent delivery system of claim 2, the stent-contacting member having an arcuate shape.

4. The stent delivery system of claim 3, the stent-contacting member forming an arc of between 20 degrees to 180 degrees.

5. The stent delivery system of claim 3, the proximal bumper further comprising a second radially-extending strut having a first end attached to the delivery member and a second end attached to the stent-contacting member, wherein the second radially-extending strut is biased to impose an outward radial force against the stent-contacting member.

6. The stent delivery system of claim 5, the stent-contacting member forming an arc of more than 180 degrees.

7. The stent delivery system of claim 2, the stent-contacting member comprising a low-friction outer surface.

8. The stent delivery system of claim 2, the radially extending strut comprising a shape-memory material.

9. The stent delivery system of claim 8, wherein the shape-memory material is nitinol.

10. The stent delivery system of claim 1, wherein the proximal bumper is configured such that, if the sheath is subject to a bending force, a cross-sectional dimension defined by the proximal bumper expands, so that the proximal bumper remains in contact with the sheath.

11. The stent delivery system of claim 1, the proximal bumper comprising a perfusion opening that permits fluid to flow from a proximal side of the proximal bumper to a distal side of the proximal bumper.

12. The stent delivery system of claim 1, the proximal bumper having a non-circular, ellipsoid cross-sectional shape.

13. The stent delivery system of claim 1, wherein the proximal bumper comprises a braided tubular member having a distal portion configured to radially expand such that, when the proximal bumper is radially constrained by the sheath, the distal portion of the braided tubular member contacts the sheath.

14. The stent delivery system of claim 1, wherein the proximal bumper comprises a tubular body portion coupled to the delivery member and a plurality of stent-contacting members extending radially outward from a distal end of the tubular body portion.

15. The stent delivery system of claim 14, wherein the tubular body and the plurality of stent-contacting members are integrally formed.

16. The stent delivery system of claim 1, further comprising a self-expanding distal bumper disposed on the delivery member distal of the stent in a radially contracted configuration, wherein the distal bumper is biased to expand to a radially expanded configuration in which the distal bumper has a cross-sectional dimension greater than an inner diameter of the sheath, such that the distal bumper, when in its radially contracted configuration, contacts the sheath and prevents movement of the stent distal of the distal bumper.

17. The stent delivery system of claim 1, further comprising a self-expanding middle bumper disposed on the delivery member within an interior of the stent in a radially contracted configuration, wherein the middle bumper is biased to expand to a radially expanded configuration, such that the middle bumper impart a radially outward force against an interior surface of the stent, thereby resisting movement of the stent relative to the delivery member.

18. The stent delivery system of claim 17, the middle bumper comprising a high friction outer coating.

19. The stent delivery system of claim 17, the middle bumper comprising a tubular body portion coupled to the delivery member and a plurality of stent-contacting members extending radially outward from a proximal end of the tubular body portion,
wherein each of the plurality of stent-contacting members is biased to expand to a radially expanded configuration, such that each of the plurality of stent-contacting members imparts a radially outward force against an interior surface of the stent, thereby resisting movement of the stent in a distal direction relative to the delivery member.

20. The stent delivery system of claim 19, wherein the tubular body and the plurality of stent-contacting members are integrally formed.
